(19) United States
(12) Patent Application Publication
(10) Pub. No.: US 2026/0118304 A1
(43) Pub. Date: Apr. 30, 2026

(54) ODOR SIGNATURE IDENTIFICATION SYSTEM

(71) Applicant: SHUBHAM CHANDRA, KATY, TX (US)

(72) Inventor: SHUBHAM CHANDRA, KATY, TX (US)

(21) Appl. No.: 18/932,529

(22) Filed: Oct. 30, 2024

(51) Int. Cl. G01N 27/04; G01N 21/25; G01N 33/00

(52) U.S. Cl. CPC G01N 27/04; G01N 21/251; G01N 33/0027; G01N 2201/127

(57) ABSTRACT

This invention introduces an advanced odor detection and classification system that integrates optical and conductive sensors with artificial intelligence for precise, real-time identification of odor profiles across diverse fields. The system employs a colorimetric sensor to capture CIELAB values and a conductive sensor to measure voltage, with data processed through a Neural Network model. This model is trained on an extensive dataset to recognize and predict odors, adapting to variations in odor intensity for enhanced accuracy. Designed for high selectivity, stability, and resilience to environmental factors, the system is ideal for applications in medical diagnostics, food quality control, environmental monitoring, and industrial safety.

### BACKGROUND

### Technical Field

The present invention relates to systems for the recognition of odor signatures and is particularly useful in various fields, including the food industry for identifying spoilage agents and different food product odors. It also plays a role in the medical industry for identifying volatile anesthetics such as ethanol and acetaldehyde, as well as precursors associated with a range of health disorders, including cancer and diabetes. Additionally, it serves the environmental industry by identifying air pollutants like benzene and toluene. This patent application and research did not involve any federally or government-sponsored research or development-based funding.

### Technical Background of the Invention

Humans detect odors through a sophisticated process involving a close interplay between the olfactory system and the brain. This process begins in the nose, specifically in a specialized area called the olfactory epithelium, a thin layer of tissue within the nasal cavity that contains olfactory receptors. These receptors are unique sensory neurons sensitive to specific molecules, called odorant molecules, which are released into the air from various substances such as food, flowers, and chemicals.

When odorant molecules enter the nasal cavity, they bind to olfactory receptors on sensory neurons based on chemical compatibility. This binding of odorants activates the receptors, each tuned to detect certain molecules depending on their chemical structure. This interaction initiates signal transduction a biochemical process that converts the chemical signal into an electrical impulse within the sensory neuron. The electrical impulse then travels along the neuron to the olfactory bulb at the brain's base, where initial processing of the smell begins, and the brain starts to identify the odor.

Once the olfactory bulb receives these signals, it relays them to other brain areas, such as the olfactory cortex and limbic system, which participate in interpreting the odor and associating it with emotions or memories. This processing in the brain produces the perception of the odor, where the brain integrates information from different olfactory receptors to determine the odor's quality (e.g., floral, fruity, pungent) and its intensity. The olfactory system's high sensitivity allows humans to detect many odorants even at low concentrations, and its close connection to memory and emotion enables specific scents to trigger vivid memories or strong emotional responses.

In essence, odor detection in humans is a remarkable blend of biological and perceptual processes, allowing us to experience and interpret the world through the sense of smell.

Humans have a highly developed but comparatively limited olfactory system, enabling them to detect a vast range of odors across thousands of unique compounds. Human odor classification typically revolves around primary categories that serve as a guide to distinguish different types of odors. These categories include fruity (non-citrus) scents, such as apples and bananas, originating from esters and ketones, and citrus scents derived from terpenes like limonene found in oranges. Floral odors, associated with flowers like roses and jasmine, are often linked to esters and alcohols, while woody scents, like pine, arise from terpenes. Spicy odors, including cloves and cinnamon, contain compounds such as eugenol, and chemical scents associated with synthetic environments are related to compounds like acetone. Lastly, minty or peppermint odors come from compounds like menthol, while sweet odors such as caramel are tied to sugars and ketones.

Humans also classify smells based on hedonic preferences (pleasant or unpleasant) and by functional categories determined by chemical composition (e.g., aldehydes and thiols). Psychophysical classifications add dimensions such as intensity (faint to strong) and quality (sharp, soft, fresh, stale). These classifications are integral to the development of odor identification technologies, providing frameworks that AI and e-nose systems can mimic for applications in diagnostics, environmental monitoring, and food safety.

Animals also possess a remarkably advanced olfactory system, with many species having highly specialized capabilities for detecting specific odors, including those related to disease. For example, dogs, with approximately 300 million olfactory receptors (compared to 6 million in humans), can detect VOCs linked to illnesses like cancer, diabetes, and infectious diseases. Their powerful olfactory processing allows them to distinguish disease-specific odors in human breath, sweat, urine, or skin. Dogs are frequently employed in medical detection for conditions such as COVID-19 and malaria.

Other animals also demonstrate impressive olfactory skills. Rats, particularly African giant pouched rats, are trained to detect tuberculosis (TB) by identifying scent markers in saliva samples, proving practical for disease detection in low-resource settings. Similarly, honeybees have shown potential in identifying diseases like tuberculosis and some cancers through a reflexive response to specific odor stimuli. Ferrets are valuable for influenza detection, and mice, with over 1,200 olfactory receptors, can identify lung cancer VOCs with high accuracy. Research into these animals' olfactory abilities has inspired biomimetic sensor technology, which seeks to replicate and even enhance these animals' olfactory capabilities in diagnostic applications.

### Odor Signature

An odor signature is the unique chemical profile of a specific scent, primarily composed of volatile organic compounds (VOCs) that together create its distinctive smell. Acting like a chemical “fingerprint,” an odor signature is characterized by the specific combination, concentration, and interaction of VOCs. Each compound, such as esters that contribute fruity notes or sulfur compounds that add pungency, plays a role in forming the core of the odor signature. The concentration ratios of these compounds are crucial, as even slight shifts in these proportions can significantly change the scent. Additional properties, such as molecular weight, polarity, and reactivity, further influence how these compounds interact and are perceived.

Odor signatures are typically captured by sensor arrays in electronic noses (e-noses), where different sensors respond to specific VOCs and create unique response patterns for each scent. These patterns are analyzed by machine learning algorithms, enabling the identification and classification of odors with precision. By recognizing these unique patterns, AI and analytical systems can use odor signatures to differentiate between similar smells, identify contaminants or spoilage, and even detect biomarkers for medical diagnostics.

### Odor Sensing Technologies

Current odor sensing technologies, often referred to as electronic noses (e-noses), are designed to detect, classify, and analyze smells by capturing the unique chemical profiles of volatile organic compounds (VOCs) in the air. These systems use various sensor types, each specialized in detecting specific VOC interactions. Metal Oxide Sensors (MOS), for instance, measure changes in electrical conductivity when gases interact with a metal oxide surface, offering high sensitivity and quick response times, making them suitable for air quality monitoring and food spoilage detection. However, they require heating elements and may experience drift over time. Conductive Polymer Sensors operate at room temperature and are popular in medical diagnostics and food quality control, though they are less stable over time.

Quartz Crystal Microbalance (QCM) Sensors detect odor molecules by measuring frequency changes in oscillating quartz crystals, providing high precision for chemical analysis and fragrance detection, though they are more costly and often need controlled environments. Surface Acoustic Wave (SAW) Sensors measure changes in acoustic wave velocity upon VOC absorption, offering quick and sensitive responses ideal for environmental monitoring and security, though they are sensitive to humidity and temperature changes. Electrochemical Sensors produce an electrical signal when gases interact with chemicals on the sensor surface, making them ideal for toxic gas detection in industrial safety applications; however, they have limited lifespans and require regular calibration.

Photoionization Detectors (PID) use ultraviolet light to ionize VOCs, creating ions that are measured as electric current, providing high sensitivity for VOCs commonly found in industrial settings, though they are limited to compounds that ionize under UV light. Optical Sensors, such as infrared or laser-based detectors, measure gas-light interactions without direct contact, offering high selectivity, though they are relatively costly and require careful calibration. Colorimetric Sensors change color upon exposure to specific chemicals, making them simple and cost-effective for food spoilage detection and air quality monitoring, though they lack the sensitivity required for broader applications.

Advanced systems, such as Biomimetic and Biohybrid Sensors, mimic natural olfactory receptors by incorporating biological elements like enzymes, offering high sensitivity for medical diagnostics. However, they often require specialized maintenance. Ion Mobility Spectrometry (IMS) detects trace compounds by measuring the movement of ionized molecules in an electric field, commonly used in security and forensics due to its high sensitivity, though it is bulky and needs skilled operation. Mass Spectrometry (MS) and Gas Chromatography-Mass Spectrometry (GC-MS) are gold standards in chemical analysis, providing detailed compound identification and quantification; however, they are costly, large, and generally limited to lab environments. Machine Learning-Enhanced E-Noses combine sensor data with machine learning algorithms to improve accuracy and adaptability, allowing real-time odor classification, although they require extensive datasets and computational power.

These odor-sensing technologies serve diverse applications across medical diagnostics, food safety, environmental monitoring, and security. Advances in machine learning and materials science are making these systems more versatile, accurate, and accessible, expanding possibilities for real-time, automated odor detection across various industries.

Table 1 highlights each technology's strengths and limitations, allowing for selection based on specific needs across applications in healthcare, food safety, environmental monitoring, and security.

TABLE 1
Summarizing the advantages and disadvantages of various odor-sensing technologies

| Odor-Sensing Technology | Advantages | Disadvantages |
|---|---|---|
| Metal Oxide Sensors (MOS) | High sensitivity, fast | Requires heating elements, prone to |
| | response | signal drift over time |
| Conductive Polymer | Operates at room | Lower stability, can degrade over |
| Sensors | temperature, detects a | time |
| | wide range of VOCs | |
| Quartz Crystal | High precision, | Expensive, requires controlled |
| Microbalance (QCM) | sensitive to minute mass | conditions |
| | changes | |
| Surface Acoustic Wave | High sensitivity, quick | Sensitive to environmental changes, |
| (SAW) | response | such as humidity and temperature |
| Electrochemical Sensors | High sensitivity and | Limited lifespan, requires regular |
| | specificity for certain | calibration |
| | gases | |
| Photoionization Detectors | High sensitivity for a | Limited selectivity, detects only |
| (PID) | wide range of VOCs | UV-ionizable compounds |
| Optical Sensors | High selectivity, non- | High cost, sensitive to calibration |
| | contact operation | |
| Colorimetric Sensors | Simple, inexpensive, | Limited sensitivity and selectivity, |
| | provides visual | often requires human interpretation |
| | indication | |
| Biomimetic and Biohybrid | High selectivity and | Expensive, complex to maintain and |
| Sensors | sensitivity | may require controlled conditions |
| Ion Mobility Spectrometry | High sensitivity and | Bulky, requires skilled operation |
| (IMS) | specificity for trace | |
| | compounds | |
| Mass Spectrometry (MS)/ | Very high sensitivity | Expensive, large, requires |
| GC-MS | and accuracy | laboratory environment |
| Machine Learning- | Improved accuracy, | Requires large datasets and |
| Enhanced E-Noses | adaptable, potential for | computational power |
| | real-time analysis | |

There are several methods used to describe smells, each with unique advantages and limitations. One popular approach is the Aroma Wheel, a visual diagram that categorizes aromas based on their chemical structure. Frequently used by perfumers, sommeliers, and food scientists, aroma wheels offer a concise way to communicate complex scent profiles. Another widely utilized method is Gas Chromatography, an analytical technique that separates the constituent odorous chemicals in a mixture. This process produces a chromatogram, which identifies individual compounds that contribute to a specific odor. Although highly informative for scientific analysis, gas chromatography often requires expert interpretation, making it less accessible to a general audience.

Sensory Mapping is another valuable technique, employing statistical analysis to map relationships between different sensory descriptors, allowing for a deeper understanding of the interactions within an odor's components. However, sensory maps can be challenging for non-experts to interpret. In more creative applications, Symbolic Representations are commonly used by artists and designers who employ colors, shapes, or textures to evoke specific aromas; for instance, yellow may suggest citrus, while purple might represent lavender. This method is particularly useful for visually conveying the essence of an aroma in art and design contexts.

Experimental Sensory Technology is also gaining attention, exploring ways to translate smells into other sensory experiences, such as sound or sight, which could revolutionize how we “experience” scents in entirely new ways. Photography, while unable to capture smells directly, evokes specific aromas by portraying scenes or objects associated with particular scents—such as an image of a freshly baked pie that brings to mind the aroma of apples and cinnamon. Additionally, Food Pairing Charts help highlight compatible food pairs based on aroma profiles, assisting chefs and culinary enthusiasts in crafting more complex and harmonious flavors.

The most effective method for visualizing an aroma often depends on the specific application. For instance, to communicate a new perfume, combining aroma wheels, symbolic representations, and photography might be ideal. Meanwhile, sensory mapping or gas chromatography may be better suited for scientific analysis or exploring the intricate relationships between compounds within an aroma.

### Odor Databases

Several odor databases support electronic nose (e-nose) research and olfactory AI systems by providing critical data on odors, sensory profiles, and molecular characteristics. Flavornet is a popular choice, containing compounds relevant to food and beverage flavors. Each entry includes compound names, odor descriptions, detection thresholds, and sensory characteristics, making it valuable for food and fragrance research. Another well-known database, the GoodScents Company Database, is widely used in the fragrance and flavor industries. It offers information on odor descriptors, molecular weights, structures, and compound sources, ideal for developing artificial noses in perfumery, flavoring, and fragrance applications.

Leffingwell's Flavor and Fragrance Materials is a commercial database with extensive information on flavor and fragrance compounds, providing odor profiles, molecular structures, and sensory data. This database is frequently used in industries focused on flavor and fragrance research, including cosmetics and food. Meanwhile, Arctander's Perfume and Flavor Chemicals, based on Steffen Arctander's comprehensive work, provides descriptions of thousands of aromatic chemicals, including both subjective odor descriptions and objective chemical data, proving highly valuable for perfumery and flavor chemistry.

The Sigma-Aldrich Flavors and Fragrances Catalog is another significant resource, offering molecular data, odor characteristics, and usage information, supporting synthetic e-nose systems in the chemical and fragrance sectors. In a more specialized context, The Pherobase focuses on pheromones and semiochemicals used for insect attraction, containing chemical structures, sources, and sensory data, making it essential for research in entomology, pest control, and pheromone-based systems. Similarly, the Perfumery Raw Materials Database is dedicated to natural and synthetic compounds used in the perfume industry, with odor profiles, molecular formulas, and relevant descriptors, supporting AI models focused on olfactory signature matching. ChEBI (Chemical Entities of Biological Interest), while not exclusively centered on odors, is a freely accessible dictionary of small chemical compounds, providing odor information alongside molecular data. It is often used as a supplementary resource for e-nose projects, especially in biological and environmental applications. The Dravnieks Odor Descriptor Database offers standardized descriptors for various compounds, supporting research focused on standardizing odor perception, which aids sensory data analysis in e-noses. Lastly, VAST (Volatile Compound Activity Search Tool) includes data on volatile compounds related to food and plant aromas, with sensory descriptors and molecular structures linked to biological activities, making it particularly useful for food aroma, plant-based fragrance research, and agricultural e-nose applications.

### Artificial Intelligence

Artificial Intelligence (AI) is a branch of computer science focused on creating systems that can perform tasks requiring human-like intelligence, such as pattern recognition, decision-making, and learning from data. Using techniques like machine learning and deep learning, AI systems analyze vast amounts of information to find patterns, make predictions, and continuously improve through experience. By leveraging algorithms, AI applications range from image and speech recognition to natural language processing, robotics, and more, enabling highly efficient and accurate automation across industries.

When it comes to odor detection, AI offers powerful capabilities for analyzing and classifying complex odor profiles. In an odor detection system (like an electronic nose or e-nose), sensor arrays capture data on volatile organic compounds (VOCs), generating unique response patterns. AI, particularly neural networks and convolutional neural networks (CNNs), processes this data to identify specific “odor signatures” with high accuracy. By training on large datasets of known odors, AI models learn to recognize subtle chemical variations, allowing them to detect specific odors associated with freshness, quality, contamination, or even disease.

In food and beverage quality control, AI can detect spoilage or changes in flavor profiles by analyzing VOC patterns, while in healthcare, AI-powered systems can non-invasively screen for diseases by detecting VOC biomarkers in human breath. Additionally, adaptive learning enables AI-based odor detection to evolve and improve, adapting to new compounds and environmental variations. This capability makes AI-driven odor detection highly versatile and efficient for applications in public health, environmental monitoring, safety, and quality control.

Together, these databases enable the development and training of AI-based e-nose systems across diverse sectors, from food and beverage industries to agriculture and pest control, enhancing the capabilities of e-nose technology in odor detection, classification, and analysis.

### CIELAB

From an image analysis perspective, CIELAB, also known as LAB color space or Lab* color space, is a color model used to represent colors in a device-independent and perceptually uniform manner. It was developed by the International Commission on Illumination (CIE) as a standard color space for accurately describing and comparing colors.

The CIELAB color model consists of three components:
L* (Lightness): The L* component represents the perceived lightness or brightness of a color. It ranges from 0 to 100, where 0 represents black and 100 represents white. The midpoint of the scale, L*=50, is considered a neutral gray.
a* (Green-Red axis): The a* component represents the position along the green-red axis. Positive values indicate a shift towards red, while negative values indicate a shift towards green. The range of a* typically extends from −128 to +127.
b* (Blue-Yellow axis): The b* component represents the position along the blue-yellow axis. Positive values indicate a shift towards yellow, while negative values indicate a shift towards blue. The range of b* typically extends from −128 to +127.

The CIELAB color space is designed to be perceptually uniform, meaning that an equal numerical difference in the Lab* values correspond to a similar perceptual difference in color across the entire color space. This makes it useful for various color-related applications, such as color management, color matching, and color difference calculations.

The hue of a color is quantified by its hue angle h_{ab} in the a*b*-plane, given in degrees (0) The hue angle of a color can be calculated from the color coordinates:

$${h}_{ab} = arctan ( \frac{{b}^{*}}{{a}^{*}} ) . \qquad Equation 1$$

Chroma is the amount of saturation of a color. Colors of high chroma are said to be clear, bright or brilliant. Dull (pastel) colors have a low chroma.

$${C}_{ab} = \sqrt{{a}^{* 2} + {b}^{* 2}} . \qquad Equation 2$$

Unlike sensors that measure physical attributes like temperature or pressure, chemical sensors are finely tuned to detect specific molecular interactions. These sensors can be grouped into two main types: discriminators and analyzers. Discriminators identify analytes based on physical properties, such as weight or vapor pressure, while analyzers go a step further, examining the chemical composition through factors like reactivity, redox potential, or acid-base interactions. But how do these sensors convert chemical interactions into measurable signals? This process, known as signal transduction, uses three primary pathways: electrical/electrochemical, thermometric, and optical—the latter being a primary focus for array components.

In biological systems, a remarkable class of molecules known as metalloporphyrins plays a central role in orchestrating some of life's most fundamental processes. These unique molecules, distinguished by a central metal ion nestled within a planar porphyrin ring, exhibit extraordinary versatility and functional diversity.

The core structure of a metalloporphyrin features a four-membered porphyrin ring, a complex macromolecule composed of nitrogen atoms linked by methine bridges. At the heart of this ring sits a metal ion-often iron, magnesium, or cobalt-whose specific identity dramatically influences the molecule's properties and functions.

One prominent example of a metalloporphyrin is heme, a critical component of hemoglobin in red blood cells and myoglobin in muscle tissues. With its central iron ion, heme is the quintessential transporter of oxygen, enabling the vital distribution of this life-sustaining molecule throughout the body. The vibrant red hue of blood is a direct result of the iron-porphyrin complex within heme.

In cellular respiration, another group of metalloporphyrins, cytochromes, plays an essential role in energy production. These iron-centered molecules facilitate electron transfer within the electron transport chain, powering the mitochondria—the cell's energy factories.

In plants and certain photosynthetic bacteria, chlorophyll, a magnesium-centered metalloporphyrin, is the driving force behind photosynthesis. This molecule acts as a biological alchemist, converting sunlight into chemical energy, thereby sustaining plant life and forming the foundation of the food chain.

The unique structure of metalloporphyrins enables them to absorb light across a broad spectrum of wavelengths, as seen in chlorophyll's function in natural photosynthesis. This property facilitates electron transfer, a crucial step in converting light energy into chemical energy. The functional versatility of metalloporphyrins is further enhanced by the ability to finely tune their properties through variations in the central metal and attached groups, allowing for tailored light absorption and electron transfer characteristics.

Beyond energy conversion, metalloporphyrins also act as efficient catalysts in enzymatic reactions, supporting the seamless progression of cellular processes. Their vibrant colors, influenced by the interplay between the central metal ion and surrounding environment, make them ideal for applications in odor sensing, where they can aid in the detection of harmful substances and deepen our understanding of chemical interactions

The choice of metalloporphyrin dye for specific odor detection applications depends on the nature of the target odor molecules. Different metalloporphyrins can be tailored or selected to exhibit specific affinities for certain classes of odor molecules or volatile compounds. Below are some examples of metalloporphyrin dyes and their potential applications for detecting specific odors:
Cobalt Porphyrins: Cobalt-based porphyrins are effective for detecting sulfur-containing compounds, which are often associated with foul odors, such as hydrogen sulfide (rotten eggs) or thiols (skunk spray). Cobalt porphyrins (Co(II) and Cu(II)) with functional groups attached to the porphyrin ring show varying affinities for water molecules, making them useful for atmospheric moisture capture and for detecting VOCs (volatile organic compounds) containing sulfur groups.
Iron Porphyrins: Iron-based porphyrins are sensitive to a broad range of odor molecules, including VOCs that contain oxygen, nitrogen, or sulfur. This makes them suitable for general odor detection and air quality monitoring across diverse environments.
Copper Porphyrins: Copper porphyrins can be tailored to interact with specific VOCs, such as aldehydes and ketones, responsible for fruity or sweet odors. They are also applicable for detecting VOCs produced during food spoilage, making them valuable in food quality monitoring.
Nickel Porphyrins: Nickel porphyrins are designed to target specific odor molecules like amines and ammonia, which are associated with fishy or pungent smells. These porphyrins are particularly useful for applications related to wastewater treatment and environmental odor control.
Manganese Porphyrins: Manganese-based porphyrins are effective in detecting nitrogen-containing compounds, such as amines and ammonia, making them suitable for monitoring industrial and agricultural odors.
Zinc Porphyrins: Zinc porphyrins offer versatile applications and can be tailored for various odor detection purposes. They are particularly suitable for detecting common odorants in household and industrial settings.
Designing and selecting the appropriate metalloporphyrin dyes for specific odors requires an in-depth understanding of the chemical interactions between the dye and target odor molecules. By fine-tuning these interactions, metalloporphyrin dyes can be optimized for specific odor detection applications, offering precision and sensitivity across a range of environmental, industrial, and consumer needs.

Conductive films for odor detection require materials capable of responding to volatile organic compounds (VOCs) or specific odor molecules by altering their electrical conductivity. Common materials used for these films include graphene and graphene oxides, carbon nanotubes, metal oxides, conducting polymers, metal nanoparticles, and doped semiconductors. Graphene and its oxidized forms are particularly effective due to their high surface area, electrical conductivity, and versatility. This material can be doped with elements or combined with nanoparticles to improve sensitivity to specific odorants, enabling a wide range of detection applications.

Carbon nanotubes (CNTs), both single-walled and multi-walled, offer exceptional sensitivity and selectivity thanks to their unique structure and conductivity. These nanotubes can be functionalized with chemical groups that make them highly responsive to particular odor molecules. Metal oxides (MOx) such as tin oxide (SnO₂), zinc oxide (ZnO), and tungsten oxide (WO₃) are also commonly used due to their reactivity to various gases, which induces noticeable conductivity changes. To improve their selectivity and response time, MOx films are often used with temperature control, making them ideal for detecting VOCs in controlled settings.

Conducting polymers, including polyaniline (PANI), polypyrrole (PPy), and polythiophene, are valued for their flexibility, lightweight nature, and the ability to tailor their responses to specific odors by adding functional groups that interact with targeted VOCs. Noble metal nanoparticles, such as gold (Au) and silver (Ag), can also be embedded in these conductive films to enhance their sensitivity and selectivity, particularly toward sulfur-containing compounds, amines, and other odor molecules. These metal nanoparticles are frequently paired with graphene or CNTs for improved response.

TABLE 2
Odorants and their corresponding Conducting Materials (sample)

| CAS No. | Molecular Weight | Odorant | Conductive Polymer |
|---|---|---|---|
| 64-19-7 | 60 | acetic acid | PEDOT:PSS |
| 67-71-0 | 90 | dimethyl sulfone, sulfonylbismethane | PEDOT:PSS |
| 75-07-0 | 44 | ethanal, acetaldehyde | PEDOT:PSS |
| 78-36-4 | 224 | linalyl butyrate, 3,7-dimethyl-1,6-octadien-3-yl butyrate | PEDOT:PSS |
| 78-93-3 | 72 | methyl ethyl ketone, 2-butanone | PEDOT:PSS |
| 80-56-8 | 136 | α-pinene, 2,6,6-trimethylbicyclo[3.1.1]hept-2-ene; cyclic dexadiene | Polyacetylene |
| 88-84-6 | 204 | (5),7(11)-diene, 1,2,3,4,5,6,7,8-octahydro-1,4-dimethyl-7-(1-methylethylidene)-a [symbol] | Polyacetylene |
| 91-10-1 | 154 | syringol, 2,6-dimethoxyphenol | Polyacetviene |
| 93-89-0 | 150 | ethyl benzoate | Polyacetylene |
| 122-78-1 | 120 | phenylethanal, phenylacetaldehyde | Polyacetylene |
| 123-32-0 | 108 | dimethyl pyrazine, 2,5-dimethyl pyrazine, 2,5-dimethyl-1,4-diazine | Polyacetylene |
| 123-72-8 | 72 | butanal | Polyacetylene |
| 97-47-3 | 226 | henylethyl benzoate, 2-phenylethyl ester benzoic acid, 2-β-phenylethyl benzoat [symbol] | Polyaniline |
| 97-89-2 | 226 | citronellyl isobutyrate, 3,7-dimethyl-6-octen-1-yl 2-methyl propenoate | Polyaniline |
| 136-60-7 | 178 | butyl benzoate | Polypyrrole |
| 140-10-3 | 148 | cinnamic acid, (E)-3-phenyl-2-propenoic acid | Polypyrrole |
| 141-27-5 | 152 | geranial, (E)-3,7-dimethyl-2,6-octadienal | Polypyrrole |
| 928-95-0 | 100 | (E)-2-hexenol, (E)-2-hexen-1-ol | Polypyrrole |
| 939-48-0 | 164 | isopropyl benzoate, 1-methylethyl benzoate | Polypyrrole |
| 1117-52-8 | 262 | [symbol] rnesylacetone, (5E,9E)-6,10,14-trimethyl-5,9,13-pentadecatrien-2-one, farnesyl a [symbol] | Polypyrrole |
| 99-48-9 | 152 | carveol, 2-methyl-5-(1-methylethenyl)-2-cyclohexenol | Polythiophene |
| 99-87-6 | 134 | p-cymene, 4-isopropyltoluene; p-methyl cumene | Polyibiophene |
| [symbol] 59121-25- [symbol] | 129 | [symbol] ronitrile, 5-(methylthio)-pentanenitrile, 1-cyano-4-(methylthio)butane, 5-methy [symbol] | Polythiophene |
| [symbol] 65505-17- [symbol] | 160 | methyldithiofurane, 2-methyl-3-methyldithio-furane | Polythiophene |
| [symbol] 67952-60- [symbol] | 150 | 2-methyl-2-(methyldithio)propanal | Polythiophene |
| [symbol] 71159-90- [symbol] | 170 | p-menthenethiol, 1-p-menthene-8-thiol | Polythiophene |
| [symbol] 74410-10- [symbol] | 151 | dill ether | Polythiophene |
| [symbol] 80041-00- [symbol] | 156 | [symbol] -whiskey lactone, 5-butyldihydro-4-methyl-2(3H)-Furanone, (−)-cis-whiskey lacto [symbol] | Polyibiophene |
| [symbol] 85213-22- [symbol] | 111 | acetylpyrroline, 2-acetyl-1-pyrroline | Polythiophene |
| [symbol] 94268-57- [symbol] | 168 | p-menthadienhydroperoxide, (E)-p-mentha-6,8-dien-2-hydroperoxide | Polythiophene |
| [symbol] 09351-28- [symbol] | 140 | 3-nonenel, (E)-3-nonenal | Polythiophene |
| [symbol] 23123-38- [symbol] | 222 | eudesmol, 7-epi-α-eudesmol | Polythiophene |
| [symbol] 33447-37- [symbol] | 125 | propionylpyrroline, 2-propionyl-1-pyrroline | Polythiophene |
| [symbol] 47159-48- [symbol] | 154 | 6-decenal, (E)-6-decenal | Polythiophene |
| [symbol] 59794-78- [symbol] | 260 | 1,5-octadienone, (E)-1,5-octadien-3-one | Polythiophene |

[symbol]

indicates data missing or illegible when filed

The field of breath analysis is in its early stages, yet it holds tremendous potential. As a non-invasive, painless, and easily accessible method, breath testing could revolutionize disease detection and monitoring, allowing for earlier intervention and improved patient outcomes. While challenges remain—such as the need to standardize breath collection and analysis methods—the future of breath analysis looks promising. Advances in research and technology are expected to make it possible to detect a growing number of diseases through a simple breath sample. Historically, scent has been used as a rudimentary diagnostic tool, but modern technology is now taking this ancient practice to new heights. Today's sophisticated instruments can analyze the complex chemical composition of exhaled breath, identifying specific volatile organic compounds (VOCs) that act as biomarkers for a variety of diseases.

Some of the key biomarkers in human breath linked to disease detection include VOCs like benzene, toluene, and ethylbenzene, which have been associated with lung cancer; acetone, isoprene, and pentane, which may be elevated in people with diabetes; and dimethyl sulfide (DMS) and other sulfur-containing VOCs, which can indicate liver disease. Additionally, nitric oxide (NO), produced in the respiratory tract, is a marker of inflammation and is associated with respiratory conditions like asthma. Carbon monoxide (CO) levels may be elevated due to environmental factors (e.g., smoking or pollution) or conditions such as chronic obstructive pulmonary disease (COPD). Other markers include ammonia, associated with liver and kidney disease, and acetone, linked to metabolic conditions such as diabetic ketoacidosis (DKA) in people with diabetes.

High levels of hydrogen and methane in breath are often linked to digestive disorders, including small intestinal bacterial overgrowth (SIBO) and irritable bowel syndrome (IBS). Isoprene, another volatile compound, is associated with oxidative stress and metabolic changes, and its levels may vary with specific diseases. Furthermore, unique breath profiles are being identified for a range of conditions, including various cancers (such as lung, breast, and colorectal), respiratory diseases, and neurological disorders like Parkinson's and Alzheimer's disease. As this technology advances, the simple act of exhaling could soon provide valuable insights into a person's health, making breath analysis a powerful tool for early diagnosis and disease monitoring.

### PRIOR ART INFORMATION

Prior art on colorimetric and conductive sensors for odor detection encompasses a range of materials, applications, and dual-mode designs, with notable advancements in sensitivity and selectivity through the integration of AI. In the realm of colorimetric sensors, metalloporphyrins (1) and metal complexes have been frequently employed for detecting specific gases such as ammonia, hydrogen sulfide, and various VOCs. These compounds react visibly with target odorants, making them effective for both visual and electronic odor detection, as widely documented in patents. pH indicators and dye-based sensors have also been commonly applied in odor detection, changing color in the presence of gases like ammonia, which is useful for both environmental monitoring and food safety. Another noteworthy area of colorimetric detection is the development of optical nanosensors with solvatochromic dyes, which shift color based on the polarity of surrounding VOCs (2). In more complex applications, microfluidic devices integrate colorimetric dyes with digital imaging to create portable odor detection solutions.

For conductive odor sensors, metal oxide semiconductors (MOx) such as tin oxide (SnO₂), zinc oxide (ZnO), and tungsten oxide (WO₃) have been widely studied for their responsiveness to various gases through conductivity changes (3). These sensors are extensively used in air quality monitoring, environmental sensing, and industrial applications. Carbon-based materials, including graphene and carbon nanotubes (CNTs), have become integral to odor detection due to their tunable surface chemistry and high conductivity, which allows selective detection of VOCs based on functionalization. Additionally, conducting polymers like polyaniline and polypyrrole offer sensitivity and flexibility, making them ideal for portable and wearable sensors in applications like real-time food freshness monitoring. Hybrid conductive films embedding metal nanoparticles (e.g., gold, silver) with materials like graphene enhance sensor response to sulfur-containing compounds, amines, and other odorants, expanding the detectable VOC range and improving sensitivity.

Innovations in dual—mode sensor designs-combining colorimetric and conductive mechanisms—have led to improved specificity and accuracy in odor detection. Such systems use a colorimetric layer that visually indicates the presence of target odorants, while a conductive layer concurrently captures changes in electrical properties. Dual-mode sensors have been developed for applications in security (e.g., explosive detection), food quality assessment, and healthcare diagnostics. Additionally, microelectromechanical systems (MEMS) with colorimetric and conductive sensor arrays are increasingly utilized to detect complex odor profiles in diverse environments, with applications in consumer products, industrial safety, and environmental monitoring (4).

Prior art also documents substantial advances in data processing for odor detection through AI integration. Machine learning models, such as neural networks, support vector machines, and principal component analysis, help interpret sensor data for accurate classification and pattern recognition, enhancing detection in challenging applications like medical diagnostics. AI models have enabled the classification of complex odor profiles, offering potential applications in early disease detection through breath analysis and forensic investigations. Innovations in calibration and drift compensation are also noted in patents, using adaptive algorithms to maintain long-term sensor accuracy under changing environmental conditions like temperature and humidity.

Across these various applications, colorimetric and conductive sensors have become essential in areas like medical diagnostics, where breath VOC analysis aids in non-invasive disease detection, and in food quality control, where spoilage compounds such as ammonia and amines are effectively monitored. Environmental monitoring also benefits from metal oxide and carbon-based sensors for detecting pollutants like carbon monoxide and benzene. The advancement of these sensors, coupled with AI, continues to improve the sensitivity, stability, and usability of odor detection systems, driving broader adoption in healthcare, environmental safety, and consumer goods.

### SUMMARY OF THE INVENTION

The invention is designed to provide advanced odor detection and classification through a combination of specialized components and AI algorithms. At its core, the system includes a Sampling System responsible for collecting and pre-processing air samples, utilizing an intake mechanism, flow control, and a pre-processing unit to ensure consistent sample quality. The Sensor Array—composed of optical and conductive sensors—detects specific odor signatures by identifying volatile organic compounds (VOCs) and other target molecules, allowing for a detailed analysis of the chemical makeup of odors.

A Data Acquisition System amplifies the signals from the sensors, converts them from analog to digital format, and performs baseline corrections, preparing the data for further analysis. This data is then processed by the Data Processing Unit, which extracts relevant features and applies pattern recognition algorithms to distinguish odor characteristics. The AI Model for Odor Classification uses a training dataset and neural network models to classify odors in real-time, offering accurate and timely predictions.

To maintain long-term accuracy, the system includes a Calibration and Drift Compensation System that utilizes calibration protocols and drift correction algorithms, countering the effects of sensor drift and environmental variations. Connectivity and Data Transmission features enable remote access and data storage by incorporating communication protocols and cloud integration, making it easy to manage and analyze data from multiple locations. Finally, the User Interface and Display provides a user-friendly interface, complete with data logging and feedback mechanisms to facilitate real-time monitoring and control of the odor detection process.

This invention is designed for precision, offering real-time, accurate odor identification for a variety of applications. Its robust sensor technology and advanced AI algorithms ensure high accuracy and stability, making it suitable for complex odor detection tasks across multiple industries.

### BRIEF DESCRIPTION OF THE DRAWINGS

Reference is made to the accompanying drawings in which is shown an illustrative embodiment of the invention, from which its novel features and advantages will be apparent.

FIG. 1 provides a simplified depiction of the Odor Signature Identification System.

FIG. 2 is a simple illustration of the different components of the Odor Signature Identification System.

FIG. 3 is an illustration of the Colorimeter sensor that allows optical analysis of the odor signature.

FIG. 4 is an illustration of the Conductive sensor that allows electrical analysis of the odor signature.

FIG. 5 is a pictorial representation of the different smell signatures.

FIG. 6 is a box graph for Molecular Weight Distribution by Odorant Class

FIG. 7 shows the CNN Model Development process for Odor Prediction

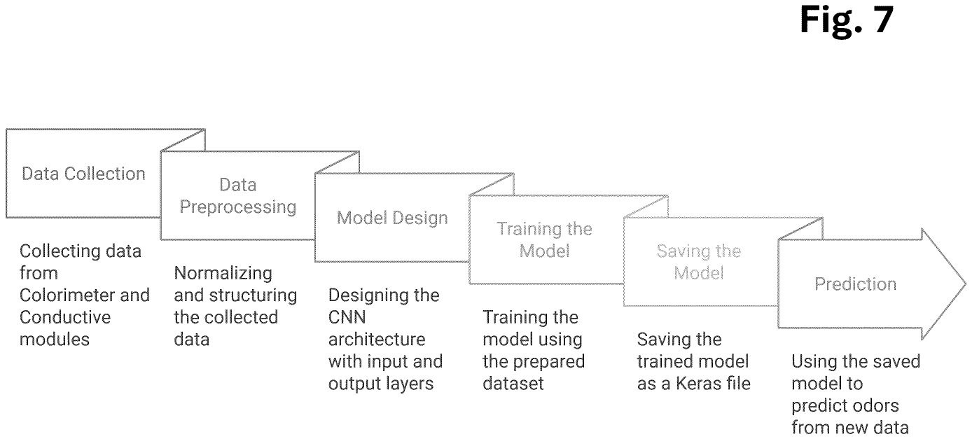

### DESCRIPTION OF THE PREFERRED EMBODIMENTS

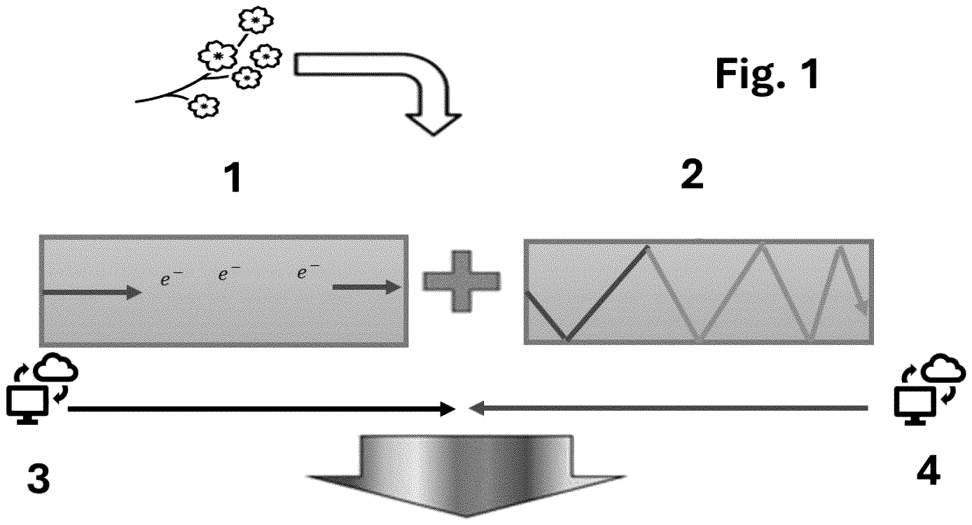

Referring to FIG. 1, it employs a combination of Electrical Analysis 1 and Optical Analysis 2. In this setup, 3 represents the AI database containing conductance data for various conducting materials. Any deviations in the conductance of these known materials, when exposed to specific odorant molecules, can be utilized for pattern recognition of the odorant molecule(s). Similarly, 4 is the AI database containing CIELAB data for different colorimetric materials. Variations in the CIELAB values of these known materials, when exposed to specific odorant molecules, can also be employed for pattern recognition of the odorant molecule(s).

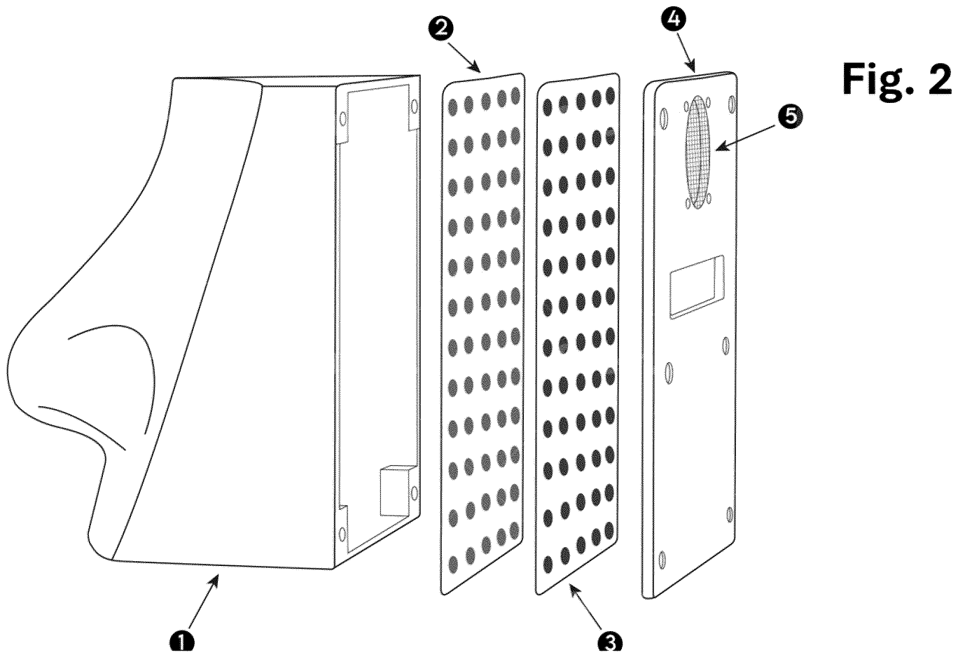

In FIG. 2, various elements of the Odor Signature Identification System are depicted. 1 represents an olfactory analyzer, while 5 is a fan that generates negative pressure, facilitating the passage of odors through the olfactory analyzer. Subsequently, the odor is introduced to 2, a colorimeter sensor, and 3, a conductive sensor. To enable the seamless integration of the olfactory sensor with 2 and 3, 4 serves as a backplate.

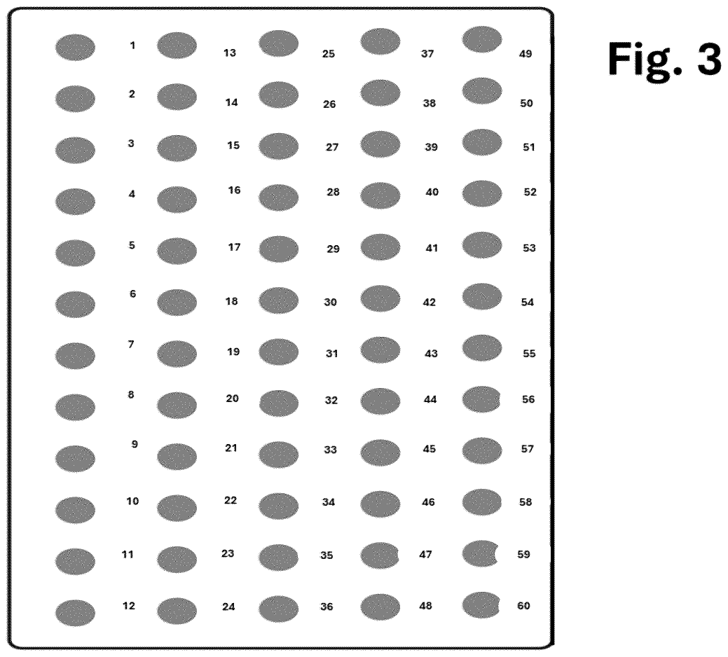

In FIG. 3, the illustration of the colorimetric sensor is shown.

Various dyes, including Metalloporphyrins, Indicative Dyes and Solvatochromic are coated on the filter to document the color fingerprints of various odorant molecules.
1 is a coating of cobalt(II) 2,3,7,8,12,13,17,18-(octaethyl)porphyrin.
2 is a coating of manganese(III) 5,10,15,20-(tetraphenyl)porphyrin chloride.
3 is a coating of nickel(II) 2,3,7,8,12,13,17,18-(octaethyl)porphyrin.
4 is a coating of nickel(II) 5,10,15,20-(tetraphenyl)porphyrin.
5 is a coating of palladium(II) 2,3,7,8,12,13,17,18-(octaethyl)porphyrin.
6 is a coating of palladium(II) 5,10,15,20-(tetraphenyl)porphyrin.
7 is a coating of platinum(II) 2,3,7,8,12,13,17,18-(octaethyl)porphyrin.
8 is a coating of platinum(II) 5,10,15,20-(tetraphenyl)porphyrin.
9 is a coating of zinc(II) 2,3,7,8,12,13,17,18-(octaethyl)porphyrin.
10 is a coating of manganese(III) 2,3,7,8,12,13,17,18-(octaethyl)porphyrin chloride.
11 is a coating of iron(III) 5,10,15,20-(tetraphenyl)porphyrin chloride.
12 is a coating of cobalt(II) 5,10,15,20-(tetraphenyl)porphyrin.
13 is a coating of copper(II) 2,3,7,8,12,13,17,18-(octaethyl)porphyrin.
14 is a coating of copper(II) 5,10,15,20-(tetraphenyl)porphyrin
15 is a coating of gallium(III) 2,3,7,8,12,13,17,18-(octaethyl)porphyrin chloride.
16 is a coating of gallium(III) 5,10,15,20-(tetraphenyl)porphyrin chloride.
17 is a coating of indium(III) 2,3,7,8,12,13,17,18-(octaethyl)porphyrin chloride.
18 is a coating of indium(III) 5,10,15,20-(tetraphenyl)porphyrin chloride.
19 is a coating of iron(III) 2,3,7,8,12,13,17,18-(octaethyl)porphyrin chloride.
20 is a coating of indium(III) 5,10,15,20-(tetraphenyl)porphyrin chloride.
21 is a coating of N,N-Dimethyl-p-phenylenediamine (DDMP)
22 is a coating of 1-(2-pyridylazo)-2-naphthol (PAN)
23 is a coating of 2,2′-azino-bis(3-ethylbenzothiazoline-6-sulfonic acid.
24 is a coating of 3,3′-Dimethoxybenzidine (DMBZ)
25 is a coating of 2,4-Dinitrophenylhydrazine.
26 is a coating of 4-Aminobipheny (4-ABP).
27 is a coating of Methylthymol blue (MTB).
28 is a coating of Phenyl Red.
29 is a coating of Alizaringelb GG.
30 is a coating of Potassium Permagnate.
31 is a coating of cobalt(II) 2,3,7,8,12,13,17,18-(octaethyl)porphyrin embedded with gold nanoparticles (2-5 nm).
32 is a coating of manganese(III) 5,10,15,20-(tetraphenyl)porphyrin chloride embedded with gold nanoparticles (2-5 nm).
33 is a coating of nickel(II) 2,3,7,8,12,13,17,18-(octaethyl)porphyrin embedded with gold nanoparticles (2-5 nm).
34 is a coating of nickel(II) 5,10,15,20-(tetraphenyl)porphyrin embedded with gold nanoparticles (2-5 nm).
35 is a coating of palladium(II) 2,3,7,8,12,13,17,18-(octaethyl)porphyrin embedded with gold nanoparticles (2-5 nm).
36 is a coating of palladium(II) 5,10,15,20-(tetraphenyl)porphyrin embedded with gold nanoparticles (2-5 nm).
37 is a coating of platinum(II) 2,3,7,8,12,13,17,18-(octaethyl)porphyrin embedded with gold nanoparticles (2-5 nm).
38 is a coating of platinum(II) 5,10,15,20-(tetraphenyl)porphyrin embedded with gold nanoparticles (2-5 nm).
39 is a coating of zinc(II) 2,3,7,8,12,13,17,18-(octaethyl)porphyrin embedded with gold nanoparticles (2-5 nm).
40 is a coating of manganese(III) 2,3,7,8,12,13,17,18-(octaethyl)porphyrin chloride embedded with gold nanoparticles (2-5 nm).
41 is a coating of iron(III) 5,10,15,20-(tetraphenyl)porphyrin chloride embedded with gold nanoparticles (2-5 nm).
42 is a coating of cobalt(II) 5,10,15,20-(tetraphenyl)porphyrin embedded with gold nanoparticles (2-5 nm).
43 is a coating of copper(II) 2,3,7,8,12,13,17,18-(octaethyl)porphyrin embedded with gold nanoparticles (2-5 nm).
44 is a coating of copper(II) 5,10,15,20-(tetraphenyl)porphyrin embedded with gold nanoparticles (2-5 nm).
45 is a coating of gallium(III) 2,3,7,8,12,13,17,18-(octaethyl)porphyrin chloride embedded with gold nanoparticles (2-5 nm).
46 is a coating of gallium(III) 5,10,15,20-(tetraphenyl)porphyrin chloride embedded with gold nanoparticles (2-5 nm).
47 is a coating of indium(III) 2,3,7,8,12,13,17,18-(octaethyl)porphyrin chloride embedded with gold nanoparticles (2-5 nm).
48 is a coating of indium(III) 5,10,15,20-(tetraphenyl)porphyrin chloride embedded with gold nanoparticles (2-5 nm).
49 is a coating of iron(III) 2,3,7,8,12,13,17,18-(octaethyl)porphyrin chloride embedded with gold nanoparticles (2-5 nm).
50 is a coating of indium(III) 5,10,15,20-(tetraphenyl)porphyrin chloride embedded with gold nanoparticles (2-5 nm).
51 is a coating of Nile Red.
52 is a coating of Sudan Red.
53 is a coating of Rhodamine Red.
54 is a coating of Phenolpthalein.
55 is a coating of Methyl Orange.
56 is a coating of Cresol Red.
57 is a coating of Bromcresol Green.
58 is a coating of o-phenylenediamine.
59 is a coating of o-phenylenediamine and methylamine.
59 is a coating of methylamine.

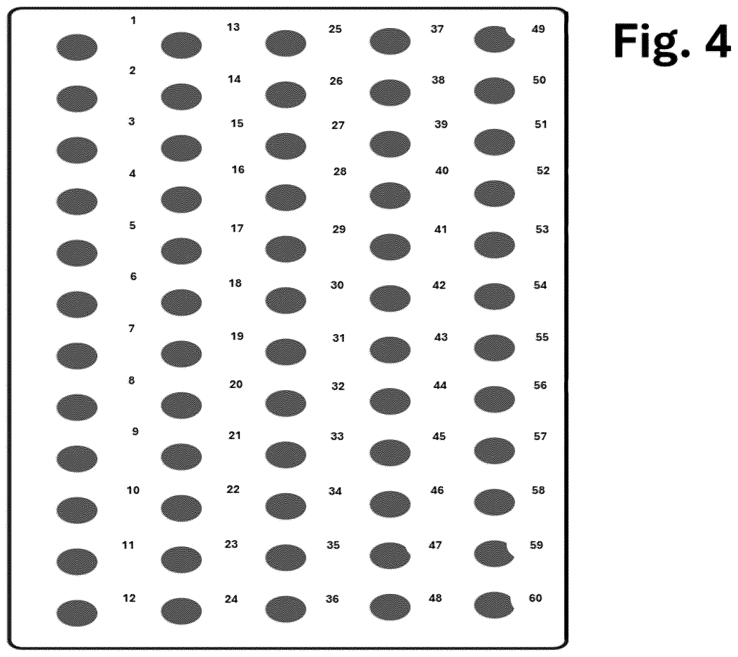

In FIG. 4, the illustration of the conductive sensor is shown.

1 to 12 is a coating of transparent graphene inks containing silver nanoparticles. 1 to 6 is graphene ink containing silver nanoparticles of ≤50 nm, 7 to 12 is graphene ink containing silver nanoparticles of >50 nm.

13 to 24 is a coating of transparent graphene inks containing gold nanoparticles. 13 to 18 is graphene ink containing gold nanoparticles of ≤10 nm, 19 to 24 is graphene ink containing gold nanoparticles of >10 nm.

25 to 36 is a coating of transparent graphene inks containing MOx including Tin Dioxide, Zinc Oxide, Indium Oxide, Tungsten Oxide, Molebdenum Oxide, Copper Oxide, Nickel Oxide and Cobalt Oxide.

37 to 48 is a coating of transparent graphene inks containing MOx and gold nanoparticles doped with Br, N, P, Ga, Cr, Mg, S, and Si.

49 to 60 is a coating of transparent graphene inks containing MOx and silver nanoparticles doped with Br, N, P, Ga, Cr, Mg, S, and Si.

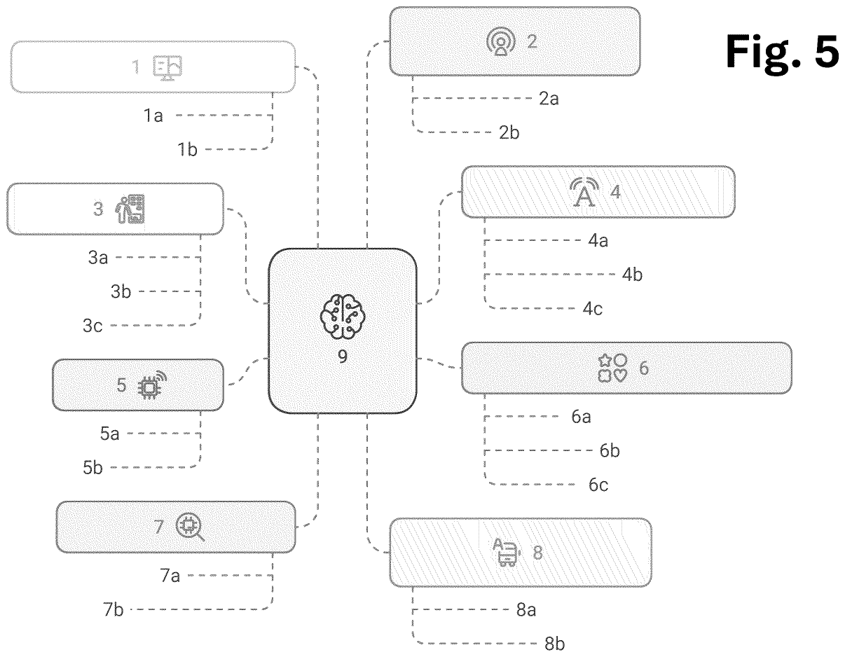

FIG. 5, illustrates the architecture of 9, Odor Signature Identification System designed for odor detection and classification. At its core, the system includes a 3, Sampling System responsible for collecting and pre-processing odor samples. This system features a 3a, intake mechanism to draw in air, 3b, flow control to maintain consistency, and a 3c, pre-processing unit that conditions samples to remove any interference, ensuring data quality. The 1, Sensor Array plays a crucial role by detecting the chemical properties of the odors. It comprises both 1a, optical and 1b, conductive sensors: the optical sensor measures colorimetric changes related to specific compounds, while the conductive sensor detects electrical conductivity variations based on the odor's composition. Data from these sensors are then processed by the 4, Data Acquisition System, which 4a, amplifies weak signals, converts them from 4b, analog to digital, and performs 4c, baseline corrections to account for potential sensor drift or environmental factors.

Following data acquisition, the 7, Data Processing Unit applies 7a, feature extraction and 7b, pattern recognition algorithms to identify key characteristics of the odors, distinguishing between different types based on unique data points. This processed data is then fed into the 6, AI Model for Odor Classification, which leverages a 6a, training dataset and 6b, neural network models to classify odors in 6c, real-time. This setup allows for immediate, accurate odor recognition by referencing a library of known odor signatures.

To ensure long-term reliability, the system includes an 8, Calibration and Drift Compensation System that adjusts for sensor aging and environmental variations, using 8a, calibration protocols and 8b, drift correction algorithms. Additionally, 2, Connectivity and Data Transmission features enable remote access and cloud storage, supported by 2a, communication protocols and 2b, cloud integration, allowing data to be managed and analyzed from various locations.

Finally, the system provides a 1, User Interface and Display for real-time monitoring, featuring a 1a, feedback mechanism for immediate results and 1b, data logging to track odor detection history. This comprehensive architecture, blending advanced sensor technology, data processing, and AI algorithms, makes the E-Nose System suitable for applications in food safety, healthcare, environmental monitoring, and beyond.

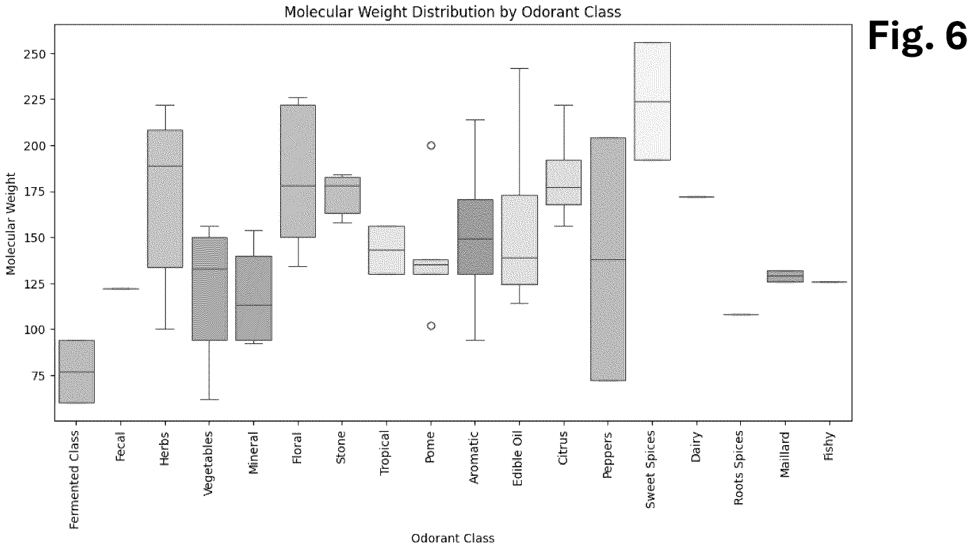

FIG. 6, is a boxplot illustrating the molecular weight distribution across different odorant classes.

The figure illustrates a step-by-step process for developing a Convolutional Neural Network (CNN) model for odor prediction. The workflow begins with Data Collection, where data is gathered from two sources: the Colorimeter Module and the Conductive Module. This stage involves capturing key metrics, including CIELAB colorimetric data from the colorimeter and voltage readings from the conductive module, across a 5×12 grid for each sample.

Following data collection, the Data Preprocessing step normalizes and structures the data, preparing it for input into the CNN. This step ensures the data is consistent and scaled, optimizing model performance and making it easier for the model to recognize patterns.

In the Model Design phase, the architecture of the CNN is configured. This involves defining the input and output layers, as well as designing convolutional and pooling layers to extract spatial patterns from the 5×12 data grids. The CNN architecture is specifically tailored to process both colorimetric and conductive data effectively.

Once the model structure is established, the Training the Model phase begins. The CNN is trained on a labeled dataset of odor profiles, allowing it to learn to associate unique colorimetric and conductivity signatures with specific odor classes. Training takes place over multiple epochs, fine-tuning the model to ensure accuracy and generalization.

After the training is complete, the model is saved in a Saving the Model step, typically as a Keras file (.h5). This file can then be loaded onto a Raspberry Pi or similar platform for real-time applications.

Finally, the Prediction phase involves using the trained model to predict odors from new, incoming data. Real-time colorimetric and conductivity data are input into the CNN, which then outputs a prediction indicating the detected odor class, displayed on an LCD or similar interface for immediate feedback. This structured process enables robust and reliable odor detection and classification across various applications.

### DETAILED DESCRIPTION OF THE INVENTION

The invention is a sophisticated odor detection and classification system that combines advanced sensor technology with artificial intelligence for real-time, precise identification of odor profiles across various applications. This system incorporates multiple components designed to work seamlessly, ensuring accurate odor analysis and adaptability to different operational environments. In operation, the odor detection system captures air samples using the Sampling System, directing the sample through both the colorimetric and conductive sensors. The resulting data is acquired, digitized, and processed to extract unique features that make up the odor's signature. The AI model compares these features to known patterns, classifying the odor in real-time. The results are displayed on the VCD, allowing users to monitor odor profiles as they are detected.

The process begins with a well-designed Sampling System that captures air samples in a controlled and consistent manner. This subsystem includes an intake mechanism to draw air, flow control to regulate the volume of the sample, and a pre-processing unit to filter out impurities. These elements ensure that the sample reaching the sensors is clean and representative of the surrounding environment, which is critical for reliable results.

At the core of the system is a Sensor Array comprising two primary types of sensors optical and conductive. The Colorimetric Sensor detects odors by capturing CIELAB color values that result from specific chemical reactions with volatile organic compounds (VOCs). This sensor is coated with color-reactive dyes that change color upon exposure to certain odorants, providing a visual print of the odor signature. In the present invention, piezoelectric inkjet printing was used to deposit transparent sol-gel films containing different metal porphyrins and phthalocyanines. These films were subsequently characterized and employed as gas sensors. The investigated compounds encompassed magnesium/manganese(III) chloride/zinc 5,10,15,20-tetraphenyl-21H,23H-porphyrin, magnesium 2,3,7,8,12,13,17,18-octaethyl-21H,23H-porphine, and zinc 2,9,16,23-tetratertbutyl-29H,31H-phthalocyanine. To create the sensing layers, porphyrin/phthalocyanine was blended with a sol-gel solution and then printed onto either glass substrates or thick paper films. These printed films were utilized to discriminate volatile organic compounds (VOCs).

In parallel, the Conductive Sensor measures changes in conductivity as odor molecules interact with sensor materials, converting these interactions into a voltage signal. The combination of these two sensors allows for a comprehensive characterization of complex odors by capturing both colorimetric and conductive responses. In the present invention, inkjet printing was used to deposit transparent graphene inks containing combination of silver nanoparticle inks (30-35 wt %, Sigma Aldrich Product No. 736473) with specific functional peptides, which can be used to identify specific odorant molecules. Previous research has indicated that electrochemical measurements indicated that specific peptides binding with the odor molecules reduces the conductivity of the graphene. The premise of the technology being, when a specific peptide binds to an odorant molecule, it can be converted into a detectable signal. The sensitivity of graphene-based sensors can be significantly improved by doping with Br, N, P, Ga, Cr, Mg, S, and Si. In addition, products such as polyaniline and polypyrrole can also be used to measure conductivity of certain odorant molecules.

An odor signature, when using the proposed sensors, combines both colorimetric and conductivity data to create a unique profile that represents the specific odor. This signature acts as a “fingerprint,” capturing the distinct interaction between the odorant and each sensor type. Here's how each component contributes to the odor signature:

The Colorimetric Component, based on the CIELAB color model, provides three essential values—L*, a*, and b*—that characterize the color response of an odor sample. The L* value represents the lightness or brightness of the color, ranging from 0 (black) to 100 (white). In the context of odor sensing, the L* value can indicate the intensity or presence of color-reactive compounds within the odor, providing insights into its overall strength. The a* value, which represents the position along the green-red axis, varies with the chemical composition of the odorant; positive values shift towards red, while negative values lean towards green, suggesting specific compounds in the odor's profile. Lastly, the b* value captures the position along the blue-yellow axis, with positive values denoting a yellow shift and negative values indicating blue. This value, similar to a*, may change in response to particular volatile organic compounds (VOCs) or other molecules within the odor, offering a comprehensive colorimetric “signature” of the odor's composition. Together, these three values enable a nuanced, quantitative understanding of the odor's color properties, which can be instrumental for accurate odor identification.

For example, an odor rich in sulfur compounds might produce CIELAB values such as L*=75, indicating a medium level of brightness, a*=−10, suggesting a slight shift towards green, and b*=30, reflecting a strong shift towards yellow. Together, these values encapsulate the colorimetric response of the odor, contributing to its unique signature. This specific combination of lightness and color shifts provides a distinctive profile that helps differentiate the sulfur-rich odor from other odors, forming a valuable part of its overall identification signature.

Conductivity Component (Voltage Response): The conductive sensor generates a voltage measurement, reflecting the conductivity of the odorant as it interacts with the sensor material (e.g., graphene or metal-oxide sensors).

Odorants with different chemical functional groups, such as sulfur, amines, or hydrocarbons, can influence the sensor's resistance or conductivity in unique ways, resulting in a distinct voltage output. For instance, an odor containing VOCs that enhance conductivity might yield a voltage reading around 3.2 volts, while an odor with more insulating compounds could reduce the voltage to approximately 1.8 volts. In a combined odor signature example, a citrus-like odor might be represented with CIELAB values of L*=80, a*=−5, and b*=40, which correspond to a light, yellowish-green tone. Alongside this colorimetric profile, the conductive sensor might record a voltage of 2.5 volts. Together, these values form a distinct odor signature for the citrus-like scent, incorporating both colorimetric and conductivity data for accurate identification and classification.

This combined signature of CIELAB values and voltage forms a reference point, which can be fed into pattern recognition and machine learning algorithms to identify and classify the odor. Each unique odor type produces a different combination of CIELAB values and voltage readings, allowing the system to effectively differentiate between various odors by recognizing their distinctive “signatures.” To measure the intensity of an odorant using CIELAB colorimetric data and conductivity voltage, a combined approach leveraging both sensor types provides a comprehensive analysis. The CIELAB colorimetric sensor measures the visual response to odorant molecules in terms of three parameters: L* (lightness), a* (green-red axis), and b* (blue-yellow axis). As the concentration of an odorant increases, it often leads to stronger color responses. This change can manifest as shifts in the CIELAB values—for instance, higher odorant concentration might lower the L* value (darker appearance) or intensify shifts along the a* and b* axes depending on the chemical characteristics of the odorant. By calibrating the sensor's CIELAB response at known odorant concentrations, the shifts in these values can be correlated with odor intensity, allowing larger changes in the colorimetric readings to indicate a higher concentration and thus greater intensity.

The conductivity sensor, on the other hand, measures the electrical response to the odorant by detecting voltage changes. As the concentration of the odorant rises, the interaction between odorant molecules and the conductive material in the sensor increases, which in turn affects the voltage output. Depending on the odorant's properties, this interaction may either increase or decrease the voltage. For example, odorants with conductive characteristics may boost the voltage with higher concentration, while those with insulating properties could cause a decrease. Through calibration with known odorant concentrations, specific voltage levels can be directly associated with odorant intensity, providing a clear scale for measurement.

Combining CIELAB and conductivity readings creates a robust intensity profile. The colorimetric data offers insights into the odorant's chemical impact on color response, while the conductivity data provides a quantitative measure of molecular interaction with the sensor. This dual approach not only allows for cross-verification—both sensors should indicate higher values or significant shifts as the odorant concentration rises—but also enhances sensitivity, as certain odorants may produce pronounced changes in only one sensor type. For instance, if measuring the intensity of a sulfurous odor, exposure to sulfur compounds might shift the colorimetric sensor toward a specific yellow-green hue, indicated by a decrease in L* and an increase in b* values, while the conductivity sensor registers an increased voltage. These combined shifts create a unique signature for the odor's intensity, enabling a precise and reliable measurement based on both CIELAB and voltage data. Together, these sensors provide a sensitive and accurate method for quantifying odor intensity.

The Data Acquisition System then processes the raw data from the sensors. This subsystem includes signal amplification to enhance sensor responses, analog-to-digital conversion for computational analysis, and baseline correction to eliminate environmental noise. The processed data is sent to the Data Processing Unit, where key features are extracted from the CIELAB and conductivity values. Pattern recognition algorithms are then applied, allowing the system to identify unique odor patterns based on the sensor data.

The processed data is fed into an AI Model for Odor Classification, where machine learning algorithms, particularly Convolutional Neural Networks (CNNs), analyze and classify the odor signature. The AI model is trained on a large dataset of labeled odor profiles, allowing it to recognize patterns linked to specific odors and even predict unknown profiles in real-time. Each odor profile in the dataset is represented across varied intensities, which enables the model to learn how changes in concentration affect the odor signature. This comprehensive training approach allows the AI to identify odors accurately, regardless of intensity fluctuations. As the model is exposed to new data, it adapts and refines its predictions, continuously increasing its reliability and accuracy over time. This adaptability is particularly useful in dynamic environments where odor intensities may vary, ensuring that the model maintains high accuracy across a wide range of applications.

Table 3 shows how a dataset for odor fingerprinting could look, including sample data for each odorant with CIELAB values and conductivity readings. This dataset can be expanded upon to train a CNN model to recognize and classify odors based on these unique odor “fingerprints.”

| | | | | Conductivity | |
|---|---|---|---|---|---|
| Odorant | L* | a* | b* | (Voltage) | Odorant Class |
| Citrus | 80.5 | −5.2 | 45.8 | .28 | Fruity |
| Lavender | 72.3 | 8.1 | −3.4 | .31 | Floral |
| Ammonia | 95.1 | −1.0 | −12.5 | .45 | Chemical |
| Coffee | 50.0 | 10.4 | 23.7 | .23 | Earthy/Herbal |
| Onion | 67.2 | −12.3 | 18.6 | .38 | Sulfuric |
| Fishy | 60.7 | −8.9 | −5.5 | .42 | Marine |
| Mint | 78.0 | −2.5 | 2.2 | .30 | Minty/Fresh |
| Vanilla | 85.0 | 4.0 | 25.0 | .27 | Sweet |
| Garlic | 65.2 | −10.5 | 16.2 | .41 | Sulfuric |
| Cheese | 55.6 | −7.8 | 14.9 | .43 | Dairy/Fermented |
| Apple | 75.5 | −2.2 | 32.6 | .26 | Fruity |
| Smoke | 45.8 | 6.7 | 5.1 | .39 | Burnt/Smoky |
| Cinnamon | 83.2 | 7.4 | 24.3 | .32 | Spicy |
| Rose | 79.5 | 8.5 | −3.7 | .31 | Floral |
| Lemon | 82.8 | −4.6 | 40.9 | .29 | Fruity/Citrus |

To ensure the system's long-term accuracy, a Calibration and Drift Compensation System is integrated, which includes protocols for regular calibration and algorithms to counter sensor drift. This feature maintains the consistency of the sensor outputs, even in changing environmental conditions. For enhanced usability, the system includes Connectivity and Data Transmission capabilities, allowing remote monitoring and data management through cloud integration and communication protocols such as Wi-Fi and Bluetooth. This feature supports large-scale deployment and enables users to access data from multiple locations.

The entire system is controlled through an intuitive User Interface and Display, featuring a visual display (VCD) that shows real-time data, including CIELAB values, conductivity readings, and classification results. The interface also provides data logging and feedback mechanisms, allowing users to monitor and adjust the system's performance as needed.

The versatility of this odor detection system makes it suitable for various applications, including quality control in the food and beverage industry, non-invasive medical diagnostics in healthcare, pollution detection in environmental monitoring, safety monitoring in industrial settings, and early pest or disease detection in agriculture. By integrating robust sensor technology with advanced AI-driven classification, this invention offers a powerful solution for real-time, precise odor detection, enabling it to handle complex odor profiles across multiple industries effectively.

Unless defined otherwise, all technical and scientific terms used herein have the same meaning as commonly understood by one of ordinary skill in the art to which this invention belongs. Although any methods and materials similar to or equivalent to those described herein can be used in the practice or testing of the present invention, example methods and materials are now described.

As used in the specification and the appended claims, the singular forms “a,” “an” and “the” include plural referents unless the context clearly dictates otherwise.

In one aspect, the present disclosure provides a method for odor signature prediction, the method comprising of conducting colorimetric and conductive assessment of the odor, and making predictions by comparing with the AI database containing conductance data for various conducting materials and AI database containing CIELAB data for different colorimetric materials.

### Advantages of the Invention

Our odor detection technology presents a significant advancement over traditional odor-sensing technologies by combining the advantages of multiple approaches while mitigating their limitations. Unlike Metal Oxide Sensors (MOS) and Surface Acoustic Wave (SAW) sensors, which require temperature controls and suffer from environmental sensitivity, our technology operates at ambient temperature and compensates for environmental fluctuations through adaptive algorithms. This is further enhanced by our drift correction system, which addresses long-term signal drift, a common drawback in MOS.

Compared to Conductive Polymer Sensors and Quartz Crystal Microbalance (QCM), our approach offers greater stability and cost-effectiveness, respectively. By using advanced coatings on conductive films, we mitigate the degradation issues seen in polymer sensors, while leveraging cost-efficient materials that achieve high precision without the expense of QCM systems. Our integrated drift compensation system extends the lifespan of the sensors, positioning it favorably against Electrochemical Sensors, which typically require frequent recalibration.

Photoionization Detectors (PID) and Optical Sensors provide high sensitivity, but with limited selectivity and higher costs, respectively. In contrast, our technology's colorimetric and conductive sensor array enables broad-spectrum detection with the added benefit of automated analysis via a Convolutional Neural Network (CNN), reducing the need for costly equipment and eliminating manual interpretation. This setup also mimics the selectivity of Biomimetic Sensors, creating a biomimetic-inspired, low-maintenance system that doesn't require the controlled conditions typical of biohybrid solutions.

Moreover, our system's compact and portable design addresses the size and operational limitations of Ion Mobility Spectrometry (IMS) and Mass Spectrometry (MS) systems, making it suitable for a wide range of field applications without the need for laboratory conditions. By leveraging machine learning-enhanced capabilities, specifically a CNN trained on diverse odorant profiles, our technology also rivals Machine Learning-Enhanced E-Noses by offering real-time predictions that are efficient and adaptable, even without extensive computational power.

Overall, our innovative odor detection system combines high selectivity and sensitivity in a portable, cost-effective solution. Its robust design, featuring automated calibration, environmental compensation, and real-time predictive capabilities, sets it apart as a versatile tool for various applications, from environmental monitoring to food quality control and beyond.

TABLE 4
ADVANTAGES OF THE INVENTION OVER OTHER TECHNOLOGIES

| Odor-Sensing | | | |
|---|---|---|---|
| Technology | Advantages | Disadvantages | Our Technology |
| Metal Oxide | High sensitivity, | Requires heating | Operates at ambient |
| Sensors (MOS) | fast response | elements, prone to | temperature, |
| | | signal drift over | reduces drift with |
| | | time | drift correction |
| | | | algorithms |
| Conductive | Operates at | Lower stability, | Enhanced stability |
| Polymer Sensors | room | can degrade over | through advanced |
| | temperature, | time | coating techniques |
| | detects a wide | | |
| | range of VOCs | | |
| Quartz Crystal | High precision, | Expensive, | Cost-effective with |
| Microbalance | sensitive to | requires controlled | environmental |
| (QCM) | minute mass | conditions | compensation |
| | changes | | algorithms |
| Surface Acoustic | High sensitivity, | Sensitive to | Adaptive |
| Wave (SAW) | quick response | humidity and | algorithms to adjust |
| | | temperature | to environmental |
| | | changes | fluctuations |
| Electrochemical | High sensitivity | Limited lifespan, | Longer lifespan |
| Sensors | and specificity | requires regular | through calibration |
| | for certain gases | calibration | and drift |
| | | | compensation |
| | | | system |
| Photoionization | High sensitivity | Limited selectivity, | Broader detection |
| Detectors (PID) | for a wide range | only detects UV- | range through |
| | of VOCs | ionizable | colorimetric and |
| | | compounds | conductive sensors |
| Optical Sensors | High selectivity, | High cost, sensitive | Integrated |
| | non-contact | to calibration | calibration |
| | operation | | protocols for |
| | | | consistent accuracy |
| Colorimetric | Simple, | Limited sensitivity | Automated analysis |
| Sensors | inexpensive, | and selectivity, | with CNN, |
| | provides visual | requires human | eliminates need for |
| | indication | interpretation | human |
| | | | interpretation |
| Biomimetic and | High selectivity | Expensive, | Mimics biomimetic |
| Biohybrid | and sensitivity | complex to | selectivity in a |
| Sensors | | maintain, | robust, low- |
| | | controlled | maintenance format |
| | | conditions required | |
| Ion Mobility | High sensitivity | Bulky, requires | Portable design |
| Spectrometry | and specificity | skilled operation | with easy-to-use |
| (IMS) | for trace | | interface |
| | compounds | | |
| Mass | Very high | Expensive, large, | Real-time analysis |
| Spectrometry | sensitivity and | requires laboratory | in a portable, cost- |
| (MS)/GC-MS | accuracy | environment | effective format |
| Machine | Improved | Requires large | CNN model |
| Learning- | accuracy, | datasets and | optimized for real- |
| Enhanced E- | adaptable, real- | computational | time odor |
| Noses | time analysis | power | prediction with |
| | potential | | efficient processing |
| | | | power |

### Examples

In this example we are developing a Convolutional Neural Network (CNN)-based odor detection system that combines colorimetric and conductive sensors, we start with hardware requirements, data collection, model training, and prediction processes. This system aims to classify odors by capturing their unique colorimetric (CIELAB) and conductive signatures and training a model to identify distinct odors, even across varying intensities.

The hardware requirements for this odor detection system consist of specialized sensors, data processing components, and supporting power and connectivity elements. The sensor array includes two primary components: a colorimetric sensor and a conductive sensor. The colorimetric sensor is a 5×12 grid coated with various metalloporphyrins, each designed to react with specific odor molecules and provide a distinct color response. To capture and convert these colorimetric changes into CIELAB values, the system uses a 5MP Arducam Mini Module Camera Shield (OV5642), which is compatible with an Arduino UNO Mega2560 board. The conductive sensor, also arranged in a 5×12 array, is coated with multiple conductive materials responsive to different odorants. This sensor captures conductivity data at each grid point, measured as voltage, to provide a complementary dataset to the colorimetric data.

For data processing, a Raspberry Pi functions as the primary platform, managing data acquisition, preprocessing, and running a pre-trained Convolutional Neural Network (CNN) model to analyze and predict odors in real time. The LCD display attached to the Raspberry Pi enables real-time visualization of predictions, providing immediate feedback to the user. The power and connectivity setup involves an adequate power source and cabling for both the Arduino and Raspberry Pi components. Additionally, the setup includes connectivity interfaces to facilitate smooth data transmission between sensors, data processing units, and the display, ensuring an efficient and integrated operation for reliable odor detection and prediction.

The data collection process involves exposing the sensor arrays to various odors at different concentrations. The fan-driven sampling system helps maintain consistent exposure, and the sensors record a set of data points that form an odor “fingerprint” for each sample. The colorimetric sensor provides three CIELAB values (L*, a*, b*) per data point, while the conductive sensor records a voltage reading at each grid point.

Each entry in the dataset corresponds to a unique odor sample, with each odor captured across different intensities to enhance the CNN model's ability to generalize.

TABLE 5
Data Table for Odor Dataset

| | Inten- | | | | | | Conduc- |
|---|---|---|---|---|---|---|---|
| Odor | sity | Sensor | Data | L* | a* | b* | tivity |
| Label | Level | Type | Point | Value | Value | Value | (Voltage) |
| Citrus | Low | Colorimetric | (0, 0) | 80 | −5 | 40 | 2.5 |
| Citrus | Medium | Colorimetric | (1, 0) | 78 | −3 | 42 | 2.7 |
| Citrus | High | Colorimetric | (2, 0) | 75 | −4 | 45 | 3.0 |
| Floral | Low | Colorimetric | (0, 1) | 65 | 10 | 20 | 2.0 |
| Sulfuric | High | Conductive | (0, 2) | — | — | — | 3.2 |

The dataset is structured such that each odor label has multiple entries representing various intensity levels, allowing the model to learn how intensity affects the odor signature.

### Training the CNN Model

The Convolutional Neural Network (CNN) model in this odor detection system is trained on a dataset that captures spatial patterns and intensity variations in CIELAB and conductivity data across multiple odor classes.

Data Preprocessing involves normalizing the CIELAB and conductivity data to a 0-1 range, optimizing model performance. The colorimetric sensor data is reshaped into a 5×12 grid format for each odor sample, with four distinct channels for the L*, a*, b* values and the conductivity voltage. This ensures compatibility with the CNN's input layer and enhances model accuracy by maintaining spatial relationships in the data.
CNN Architecture: The model architecture comprises multiple convolutional layers that extract spatial patterns from the sensor grid data, followed by pooling layers that reduce data dimensionality and emphasize key features. These layers are followed by dense (fully connected) layers that capture higher-order relationships between the sensor responses and specific odor classes. The output layer is a softmax layer, which assigns probability scores to each odor class, enabling the model to make accurate classifications.
Model Compilation and Training: The CNN model is compiled using categorical cross-entropy as the loss function and the Adam optimizer, which facilitates efficient weight updates. The training process spans multiple epochs, with a validation dataset monitoring model performance and preventing overfitting. The CNN learns to associate unique CIELAB and conductivity profiles with each odor class, enabling it to differentiate odors and adapt to various intensity levels.
Saving the Model: After training, the model is saved as a .h5 Keras file, which is compatible with the Raspberry Pi. This allows the model to be easily loaded and deployed for real-time odor detection.

### Google Colab Based Code for Training

from tensorflow.keras.models import Sequential
from tensorflow.keras.layers import Conv2D, MaxPooling2D, Flatten, Dense, Dropout

```
  def build_cnn_model( ):
    model = Sequential( )
    # First convolutional layer
    model.add(Conv2D(32, (3, 3), activation=‘relu’,
    input_shape=(5, 12, 4)))
    model.add(MaxPooling2D((2, 2)))
    # Second convolutional layer
    model.add(Conv2D(64, (3, 3), activation=‘relu’))
    model.add(MaxPooling2D((2, 2)))
    # Flatten the output
    model.add(Flatten( ))
    # Fully connected layer
    model.add(Dense(128, activation=‘relu’))
    model.add(Dropout(0.5)) # Adding dropout for regularization
    # Output layer - number of classes based on unique odors
    model.add(Dense(num_classes, activation=‘softmax’))
    return model
  # Compile the model
  model = build_cnn_model( )
model.compile(optimizer=‘adam’, loss=‘categorical_crossentropy’,
metrics=[‘accuracy’])
```

Once deployed on the Raspberry Pi (coupled with an operational amplifier), the model performs real-time predictions through a streamlined workflow involving data acquisition, preprocessing, and prediction.

During Data Acquisition, the system's fan directs the odor sample over the sensors. The Arducam captures the colorimetric data, while the conductivity sensor generates a 5×12 voltage matrix reflecting the odorant's conductive properties. These data points provide a comprehensive snapshot of the odor's chemical profile.

In the Preprocessing phase, the Raspberry Pi normalizes the real-time data to ensure consistency with the format expected by the CNN model. The data is reshaped to fit the CNN's input structure, maintaining the spatial configuration of the sensor grid to enhance the model's accuracy in recognizing patterns specific to different odor profiles.

Finally, in the Odor Prediction phase, the processed data is fed into the CNN, which then outputs a probability distribution for each odor class. The class with the highest probability is identified as the detected odor, and this result is displayed on an LCD screen for immediate feedback. This seamless, real-time feedback loop allows users to obtain accurate odor identification results instantly.

### Google Colab Based Code for Prediction

# Import necessary libraries
import numpy as np
import tensorflow as tf
from tensorflow.keras.models import load_model
import matplotlib.pyplot as plt
# Example colorimetric and conductive sensor data
# Assuming 5x12 grid for both sensors; simulated data for demonstration
# Colorimetric data will contain CIELAB values; each grid point has 3 values [L, a, b]
colorimetric_data = np.random.randint(0, 100, size=(5, 12, 3)) # Random CIELAB values
# Conductive sensor data will have voltage values across the 5x12 grid
conductive_data = np.random.uniform(1.5, 3.5, size=(5, 12)) # Simulated voltage data
# Load pre-trained CNN model (ensure to upload or link your Keras model file here)
# Assuming a model saved as ‘odor_classification_model.h5’
model = load_model(‘odor_classification_model.h5’)
# Process colorimetric data: flatten and normalize CIELAB values for CNN input
colorimetric_flattened = colorimetric_data.reshape(5*12, 3) / 100.0 # Normalize L, a, b values
to [0, 1]
# Process conductive data: flatten and normalize voltage readings
conductive_flattened = conductive_data.reshape(5*12, 1) / 3.5 # Assuming max voltage is
3.5V for normalization
# Combine colorimetric and conductive data into a single feature array
# Shape will be (5*12, 4) where 4 = 3 (CIELAB) + 1 (voltage)
sensor_data_combined = np.hstack((colorimetric_flattened, conductive_flattened))
# Reshape data for CNN model input
# Assuming model expects input shape (height, width, channels) for CNN
input_data = sensor_data_combined.reshape(1, 5, 12, 4) # Reshape to match expected input
dimensions
# Make prediction with the model
predicted_class = model.predict(input_data)
predicted_class_index = np.argmax(predicted_class)
# Display prediction
print(f“Predicted Odor Class Index: {predicted_class_index}”)
print(f“Confidence Scores: {predicted_class}”)

Humans and animals differ greatly in their odor-detecting capabilities, both in terms of sensitivity and the range of detectable compounds. While humans can detect thousands of odors, our sense of smell is relatively limited compared to many animals, particularly mammals like dogs and rodents. These animals have far more olfactory receptor genes, allowing them to detect a broader spectrum of compounds at much lower concentrations. For example, dogs possess around 1,000 functional olfactory receptor genes, while humans have only about 400. This genetic diversity and increased receptor range enable animals to pick up complex, nuanced scents, from minute traces of explosives to subtle pheromones.

Incorporating insights from the olfactory genes of animals into odor-detection technology, particularly in a CNN-based model, could significantly improve the system's sensitivity and scope. By studying the olfactory receptor genes from animals, researchers can identify specific receptors responsible for detecting various chemical classes and volatile organic compounds (VOCs). With this information, synthetic sensors can be engineered to mimic the selectivity and range of animal olfactory receptors. For instance, creating sensor arrays that replicate the genetic patterns of dogs' or rodents' olfactory systems would allow the CNN model to process a more extensive range of odor signatures, including those beyond human detection thresholds.

For CNN model training, using this expanded set of data from biologically inspired sensor responses enables the model to learn from a broader variety of odor profiles. This means that the CNN could be exposed to a larger and more diverse dataset, encompassing odorant molecules detectable by both human and animal receptors. This would enhance the CNN's ability to classify subtle and complex odors, thereby expanding its practical applications. By mimicking the genetic approach of animal olfaction, the odor detection system could achieve higher accuracy in fields like medical diagnostics, food safety, and environmental monitoring, where the detection of trace odorants is critical.

### Commercial Applications

Odor Signature Identification Systems have diverse commercial applications, enabling precise odor detection, classification, and analysis for quality control, diagnostics, and safety across industries. In medical diagnostics, these systems offer non-invasive disease detection by identifying VOCs linked to conditions like cancer, diabetes, and infections such as COVID-19, with particular value in continuous patient monitoring, especially for remote or intensive care settings. Within the food and beverage industry, odor identification systems play a vital role in ensuring product freshness and quality by detecting spoilage-related VOCs and maintaining flavor consistency across batches, which is crucial for sectors like dairy, meat, wine, and coffee. Environmental monitoring is another key area, where odor identification systems detect pollutants, hazardous chemicals, and toxic gases to maintain air quality and regulatory compliance in industries like waste treatment and manufacturing. In agriculture, these systems identify early signs of pest infestations or plant diseases via odor signatures, optimizing crop health and yields while also assessing produce ripeness for optimal storage and distribution. The cosmetics and personal care industry relies on these systems to ensure fragrance consistency in perfumes and screen products for contaminants, safeguarding product quality and consumer safety.

For security and forensics, odor identification technology aids in explosives and narcotics detection, supporting law enforcement and airport security, while forensic investigators use it in cases involving accelerants or decomposition detection. In automotive applications, odor systems improve cabin air quality and help detect maintenance issues like fuel leaks or overheating. Smart home and consumer electronics integrate odor sensors for indoor air quality monitoring and appliance functionality, detecting issues such as food spoilage or harmful chemicals.

Additionally, in textile and material production, odor signature systems monitor emissions during manufacturing to ensure products are odor-neutral and safe, and in packaging, they verify that materials do not impart unwanted odors to enclosed products. Across these applications, Odor Signature Identification Systems are invaluable for quality assurance, regulatory compliance, safety, and enhanced customer satisfaction, proving essential in sectors from healthcare and food safety to forensics and consumer goods.

In space exploration, Odor Signature Identification Systems are critical for safety, environmental monitoring, and health diagnostics for astronauts. In the confined and isolated environment of spacecraft, maintaining air quality is vital; these systems continuously monitor for harmful gases, contaminants, or unexpected chemicals, ensuring a safe atmosphere for crew members. By detecting VOCs emitted by materials, machinery, or potential leaks, odor identification systems can alert astronauts and ground control to emerging issues like coolant or fuel leaks before they become hazardous.

Odor identification also supports life-support systems by monitoring and balancing oxygen, carbon dioxide, and other gases in closed habitats, especially on long-duration missions on the ISS or planned lunar and Martian bases. These systems ensure stable environmental conditions, identifying deviations that might indicate issues with recycling or filtration equipment. For crew health, odor signature detection can monitor astronaut well-being by analyzing VOCs in breath or skin emissions that signal stress, fatigue, or potential illness. This non-invasive health monitoring is especially valuable for extended missions, where early detection of medical issues is crucial given limited access to immediate healthcare. In terms of food safety, odor systems can detect food spoilage in real time, ensuring consumables remain safe over long storage periods, which is critical for missions with limited resupply options.

As space exploration progresses toward lunar bases and Mars missions, odor signature identification systems will be essential for maintaining habitat safety and quality control. They will support sustainable living environments by monitoring contaminants, preserving air quality, and safeguarding astronaut health, ultimately contributing to mission success and crew well-being in extraterrestrial habitats.

The technology used in odor identification systems shares significant overlap with flavor detection, as both rely on sensing volatile organic compounds (VOCs) and capturing unique chemical profiles. In the human experience, flavor is a complex interplay between smell and taste, with olfactory perception playing a crucial role in identifying flavor notes. By adapting odor detection technology, which captures these VOCs, we can develop a sophisticated flavor identification system that incorporates both olfactory and taste elements.

Odor and flavor detection systems both rely on core sensor mechanisms, such as colorimetric and conductive sensors. In an odor identification setup, colorimetric sensors measure CIELAB values to capture color changes caused by odorants, while conductive sensors record voltage changes in response to VOCs. These principles can be extended to flavor detection by integrating sensors that detect compounds associated with taste (e.g., sweetness, bitterness, saltiness). For example, acids or sugars might produce distinct colorimetric responses, while conductive sensors could capture variations in conductivity for ions linked to savory flavors.

Pattern recognition and AI-based classification, key to odor identification, are similarly applicable to flavor profiling. By training a Convolutional Neural Network (CNN) on a dataset that combines data from both smell and taste sensors, the model can learn to recognize comprehensive “flavor signatures” that reflect both aroma and taste components. This allows for the identification of specific flavor profiles and intensities, making the system versatile across different applications. The applications of a flavor identification system are vast. In food and beverage production, such a system could monitor quality, detect spoilage, and ensure consistency. It could also facilitate flavor innovation by analyzing and replicating precise flavor profiles, aiding product development. Additionally, this technology could support healthcare by improving the palatability of medications through flavor masking.

In essence, the advanced sensing and machine learning capabilities of an odor identification system can be readily adapted to flavor profiling, offering transformative potential across food, beverage, and healthcare industries. This cross-application of odor detection technology provides an efficient, accurate, and scalable approach to understanding and controlling flavor.

1: An odor detection and classification system comprising:
a colorimetric sensor configured to capture color data in response to odorant interactions;
a conductive sensor configured to detect odorant interactions by measuring electrical conductivity, represented as a voltage response;
a data processing unit configured to receive, preprocess, and normalize data from both the colorimetric and conductive sensors, with the processed data formatted to match the input requirements of a convolutional neural network (CNN) model;
a CNN model trained on a dataset comprising both colorimetric and conductivity data associated with various odor profiles to classify the odor in real-time;
an output display configured to present the classified odor based on predictions from the CNN model.

2: The odor detection and classification system of claim 1, wherein the colorimetric sensor captures color data that creates a unique profile for each odorant.

3: The odor detection and classification system of claim 1, wherein the CNN model is trained using a dataset that includes variations in odorant intensity for both colorimetric and conductive data, allowing adaptive classification for different odor intensities.

4: The odor detection and classification system of claim 1, further comprising a calibration unit configured to perform periodic drift compensation on both colorimetric and conductive sensors to ensure consistent performance over time.

5: The odor detection and classification system of claim 1, wherein the CNN model is trained using a dataset with odorant intensity variations for both colorimetric and conductive data, allowing adaptive classification based on odor intensity.

6: The odor detection and classification system of claim 1, wherein the data processing unit performs baseline correction on both colorimetric and conductivity data prior to CNN input to enhance prediction accuracy.

7: The odor detection and classification system of claim 1, wherein the CNN model outputs a probability distribution across odor classes, identifying the class with the highest probability as the detected odor.

8: The odor detection and classification system of claim 1, wherein the CNN model provides a probability distribution for each odor class, selecting the class with the highest probability as the identified odor.

9: The odor detection and classification system of claim 1, further comprising memory storage for saving the CNN model in Keras format, enabling deployment on external processing devices.

10: The odor detection and classification system of claim 1, wherein the output display is configured to show real-time odor classification, intensity levels, and sensor status, facilitating user interaction and monitoring.

11: An odor detection and classification system comprising:
a colorimetric sensor configured to detect odorant interactions by capturing color data;
a data processing unit configured to receive and preprocess the color data from the colorimetric sensor;
a CNN model trained on a dataset of odor profiles to classify the odor based on the color data; and
an output display configured to present the classified odor in real-time based on the CNN model prediction.

12: An odor detection and classification system comprising:
a conductive sensor configured to detect odorant interactions by measuring electrical conductivity in terms of voltage response;
a data processing unit configured to receive and preprocess voltage data from the conductive sensor;
a convolutional neural network (CNN) model trained on a dataset of odor profiles to classify the odor based on the conductivity data; and
an output display configured to present the classified odor in real-time based on the CNN model prediction.

13: The odor detection and classification system of claim 11, wherein the CNN model is trained on a dataset that includes varying odorant intensity levels for enhanced adaptive classification.

14: The odor detection and classification system of claim 11, further comprising a calibration unit to periodically calibrate the colorimetric sensor, ensuring accuracy under different environmental conditions.

15: The odor detection and classification system of claim 11, wherein the data processing unit is configured to normalize the color data before inputting it into the CNN model, thereby enhancing consistency in predictions.

16: The odor detection and classification system of claim 12, wherein the conductive sensor includes materials sensitive to a range of volatile organic compounds (VOCs) to measure conductivity response specific to each odorant.

17: The odor detection and classification system of claim 12, wherein the CNN model is trained on a dataset that includes conductivity data for various odorants at different intensity levels, allowing for adaptive classification based on odor intensity.

18: The odor detection and classification system of claim 12, wherein the data processing unit performs baseline correction on the voltage data to enhance accuracy in sensor response.

19: The odor detection and classification system of claim 12, further comprising a calibration unit configured to adjust the conductivity baseline for environmental drift compensation.

20: The odor detection and classification system of claim 12, wherein the conductive sensor includes a range of conductive materials selected for high sensitivity to sulfur-containing compounds, amines, and other specific odorant groups.

21: The odor detection and classification system of claim 12, wherein the CNN model is trained using a dataset of conductivity measurements from the conductive sensor, processed and labeled to ensure robust odor classification across a variety of compounds.
